SEQUENCE LISTING (66 sequences)

SEQ ID NO: 1 - DNA, 46 bp, Artificial
  GSTP1 Scorpion
  modified_base (23)..(23): -BHQ-HEG-
  modified_base (23)..(23): -BHQ (Black Hole Quencher) -HEG (Hexaethylglycol) -
ccccgaacgtcgaccgctcggggcgatttcggggattttagggcgt

SEQ ID NO: 2 - DNA, 21 bp, Artificial
  GSTP1 Scorpion Antisense Primer
aaaatcccgcgaactcccgcc

SEQ ID NO: 3 - DNA, 46 bp, Artificial
  GSTP1 Scorpion
  modified_base (23)..(23): -BHQ-HEG-
cccgaacgtcgaccgctttcgggcgatttcggggattttagggcgt

SEQ ID NO: 4 - DNA, 21 bp, Artificial
  GSTP1 Scorpion Antisense Primer
aaaatcccgcgaactcccgcc

SEQ ID NO: 5 - DNA, 44 bp, Artificial
  GSTP1 Scorpion
  modified_base (22)..(22): -BHQ-HEG-
cggcgggagttcgcgggcgccgactaaatcacgacgccgaccgc

SEQ ID NO: 6 - DNA, 22 bp, Artificial
  GSTP1 Scorpion Antisense Primer
cggttagttgcgcggcgatttc

SEQ ID NO: 7 - DNA, 41 bp, Artificial
  GSTP1 Scorpion
  modified_base (19)..(19): -BHQ-HEG-
  modified_base (25)..(25): -BHQ-HEG-
cgggagttcgcgggtcccgactaaatcacgacgccgaccgc

SEQ ID NO: 8 - DNA, 22 bp, Artificial
  GSTP1 Scorpion Antisense Primer
cggttagttgcgcggcgatttc

SEQ ID NO: 9 - DNA, 50 bp, Artificial
  GSTP1 Scorpion
  modified_base (27)..(27): -BHQ-HEG-
gtggttgatgtttggggtatcaaccacaatcccacaaactcccaccaacc

SEQ ID NO: 10 - DNA, 27 bp, Artificial
  GSTP1 Scorpion Antisense Primer
gtggtgattttggggattttagggtgt

SEQ ID NO: 11 - DNA, 47 bp, Artificial
  GSTP1 Scorpion
  modified_base (24)..(24): -BHQ-HEG-
accccagtggttgatgtttggggtaatcccacaaactcccaccaacc

SEQ ID NO: 12 - DNA, 27 bp, Artificial
  GSTP1 Scorpion
gtggtgattttggggattttagggtgt

SEQ ID NO: 13 - DNA, 56 bp, Artificial
  GSTP1 Scorpion
  modified_base (28)..(28): -BHQ-HEG-
ccccacaggttggtgggagtttgtggggcccaatactaaatcacaacaccaaccac

SEQ ID NO: 14 - DNA, 27 bp, Artificial
  GSTP1 Scorpion Antisense Primer
tggttagttgtgtggtgattttgggga

SEQ ID NO: 15 - DNA, 46 bp, Artificial; identical to SEQ ID NO: 1
  GSTP1 Scorpion
  modified_base (23)..(23): -BHQ-HEG-

SEQ ID NO: 16 - DNA, 21 bp, Artificial
  GSTP1 Scorpion Antisense Primer
aaaatcccgcgaactcccgcc

SEQ ID NO: 17 - DNA, 53 bp, Artificial
  GSTP1 Scorpion
  modified_base (30)..(30): -BHQ-HEG-
cgcacgccgaacgtcgaccgcaaacgtgcgcgatttcggggattttagggcgt

SEQ ID NO: 18 - DNA, 21 bp, Artificial
  GSTP1 Scorpion Antisense Primer
aaaatcccgcgaactcccgcc

SEQ ID NO: 19 - DNA, 47 bp, Artificial
  GSTP1 Scorpion
  modified_base (27)..(27): -BHQ-HEG-
cgcacggcgaactcccgccgacgtgcgtgtagcggtcgtcggggttg

SEQ ID NO: 20 - DNA, 22 bp, Artificial
  GSTP1 Scorpion Antisense Primer
gccccaatactaaatcacgacg

SEQ ID NO: 21 - DNA, 55 bp, Artificial
  GSTP1 Scorpion
  modified_base (34)..(34): -BHQ-HEG-
ccgacgcacaaaaaaacaccctaaaatccgtcggggttagttgtgtggtgatttt

SEQ ID NO: 22 - DNA, 20 bp, Artificial
  GSTP1 Scorpion Antisense Primer
cacaacaccaaccactcttc

SEQ ID NO: 23 - DNA, 29 bp, Artificial
  GSTP1 Taqman Primer
cgtgatttagtattggggcggagcggggc

SEQ ID NO: 24 - DNA, 25 bp, Artificial
  GSTP1 Taqman Primer
atccccgaaaaacgaaccgcgcgta

SEQ ID NO: 25 - DNA, 26 bp, Artificial
  GSTP1 Taqman Probe
tcggaggtcgcgaggttttcgttgga

SEQ ID NO: 26 - DNA, 48 bp, Artificial
  RARB2 Scorpion
  modified_base (28)..(28): -BHQ-HEG-
gccgccgtcgagaacgcgagcgggcggctaccccgacgatacccaaac

SEQ ID NO: 27 - DNA, 27 bp, Artificial
  RARB2 Scorpion Antisense Primer
gggattagaattttttatgcgagttgt

SEQ ID NO: 28 - DNA, 47 bp, Artificial
  RARB2 Scorpion
  modified_base (27)..(27): -BHQ-HEG-
cggcaggattgggatgtcgagctgccgtaccccgacgatacccaaac

SEQ ID NO: 29 - DNA, 27 bp, Artificial
  RARB2 Scorpion Antisense Primer
gggattagaattttttatgcgagttgt

SEQ ID NO: 30 - DNA, 49 bp, Artificial
  RASSFIA Scorpion
  modified_base (25)..(25): -BHQ-HEG-
gccgcggtttcgttcggttcgcggccccgtacttcgctaactttaaacg

SEQ ID NO: 31 - DNA, 18 bp, Artificial
  RASSFIA Scorpion Antisense Primer
gcgttgaagtcggggttc

SEQ ID NO: 32 - DNA, 48 bp, Artificial
  TIMP3 Scorpion
  modified_base (26)..(26): -BHQ-HEG-
gcggcgagttcgggttgtagcgccgccgcctctccaaaattaccgtac

SEQ ID NO: 33 - DNA, 21 bp, Artificial
  TIMP3 Scorpion Antisense Primer
gcgtcggaggttaaggttgtt

SEQ ID NO: 34 - DNA, 44 bp, Artificial
  APC Scorpion
  modified_base (25)..(25): -BHQ-HEG-
gccggcgggttttcgacgggccggccgaaccaaaacgctcccca

SEQ ID NO: 35 - DNA, 25 bp, Artificial
  APC Scorpion Antisense Primer
gtcggttacgtgcgtttatatttag

SEQ ID NO: 36 - DNA, 44 bp, Artificial
  Actin Scorpion
  modified_base (22)..(22): -BHQ-HEG-
gcgcccaaccgcacaagggcgcgggtatattttcgaggggtacg

SEQ ID NO: 37 - DNA, 18 bp, Artificial
  ACTIN Scorpion Antisense Primer
cgacccgcactccgcaat

SEQ ID NO: 38 - DNA, 52 bp, Artificial
  ACTIN Scorpion
  modified_base (27)..(27): -BHQ-HEG-
ccgcgcatcaccaccccacacgcgcggggagtatataggttggggaagtttg

SEQ ID NO: 39 - DNA, 27 bp, Artificial
  ACTIN Scorpion Antisense Primer
aacacacaataacaaacacaaattcac

SEQ ID NO: 40 - DNA, 50 bp, Artificial
  ACTIN Scorpion
  modified_base (27)..(27): -BHQ-HEG-
cccggctaaacccaccatccagccggggggagggtagtttagttgtggtt

SEQ ID NO: 41 - DNA, 29 bp, Artificial
  ACTIN Scorpion Antisense Primer
caaaacaaaaaaactaaatctacacaacc

SEQ ID NO: 42 - DNA, 47 bp, Artificial
  ACTIN Scorpion
  modified_base (26)..(26): -BHQ-HEG-
ccgcggaacattcaactcaaccgcggggaggaggaaggtaggttttt

SEQ ID NO: 43 - DNA, 29 bp, Artificial
  ACTIN Scorpion Antisense Primer
acatacaacaatcaataacataaaaccac

SEQ ID NO: 44 - DNA, 47 bp, Artificial
  PTGS2/COX2 Scorpion
  modified_base (22)..(22): -BHQ-HEG-
cacgccgccgtatctaggcgtggtttgtttcgacgtgattttttcga

SEQ ID NO: 45 - DNA, 21 bp, Artificial
  PTGS2/COX2 Antisense Primer
gcaaaaaatcccctctcccgc

SEQ ID NO: 46 - DNA, 45 bp, Artificial
  PTGS2/COX2 Scorpion
  modified_base (22)..(22): -BHQ-HEG-
gccgcgcacaaatttccgcggcgaattggttttcggaagcgttcg

SEQ ID NO: 47 - DNA, 16 bp, Artificial
  PTGS2/COX2 Antisense Primer
cccgaattccaccgcc

SEQ ID NO: 48 - DNA, 46 bp, Artificial
  PTGS2/COX2 Scorpion
  modified_base (23)..(23): -BHQ-HEG-
ggcggaacgcacaaatttccgccgaattggttttcggaagcgttcg

SEQ ID NO: 49 - DNA, 16 bp, Artificial
  PTGS2/COX2 Antisense Primer
cccgaattccaccgcc

SEQ ID NO: 50 - DNA, 51 bp, Artificial
  PTGS2/COX2 Scorpion
  modified_base (26)..(26): -BHQ-HEG-
tgccgccgccgtatctaatggcggcagtttgtttcgacgtgattttttcga

SEQ ID NO: 51 - DNA, 21 bp, Artificial
  PTGS2/COX2 Antisense Primer
gcaaaaaatcccctctcccgc

SEQ ID NO: 52 - DNA, 51 bp, Artificial
  14-3-3 Scorpion
  modified_base (26)..(26): -BHQ-HEG-
cggccttcgctcttcgcaaaaggccgggtagtttttatgaaaggcgtcgtg

SEQ ID NO: 53 - DNA, 18 bp, Artificial
  14-3-3 Antisense Primer
ctaaccgcccaccacgtt

SEQ ID NO: 54 - DNA, 4260 bp, Homo sapiens
  misc_feature: >gi|341173|gb|M24485.1|HUMGSTP1G Homo sapiens (clone pHGST-pi) glutathione S-transferase pi (GSTP1) gene, complete cds
aacaagagatcaatatctagaataaatggagatctgcaaatcaacagaaagtaggcagcaaagccaaagaaaatagcctaaggcacagccactaaaaggaacgtgatcatgtcctttgcagggacatgggtggagctggaagccgttagcctcagcaaactcacacaggaacagaaaaccagcgagaccgcatggtctcacttataagtgggagctgaacaatgagaacacatggtcacatggcggcgatcaacacacactggtgcctgttgagcggggtgctggggagggagagtaccaggaagaatagctaagggatactgggcttaatacctgggtgatgggatgatctgtacagcaaaccatcatggcgcacacacctatgtaacaaacctgcacatcctgcacatgtaccccagaacttcaaataaaagttggacggccaggcgtggtggctcacgcctgtaatcccagcactttgggaagccgaggcgtgcagatcacctaaggtcaggagttcgagaccagcccggccaacatggtgaaaccccgtctctactaaaaatacaaaaatcagccagatgtggcacgcacctataattccacctactcgggaggctgaagcagaattgcttgaacccgagaggcggaggttgcagtgagccgccgagatcgcgccactgcactccagcctgggccacagcgtgagactacgtcataaaataaaataaaataacacaaaataaaataaaataaaataaaataaaataaaataataaaataaaataaaataaaataaaataaaataaaataaagcaatttcctttcctctaagcggcctccacccctctcccctgccctgtgaagcgggtgtgcaagctccgggatcgcagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgctccccggggatggggctcagagctcccagcatggggccaacccgcagcatcaggcccgggctcccggcagggctcctcgcccacctcgagacccgggacgggggcctaggggacccaggacgtccccagtgccgttagcggctttcagggggcccggagcgcctcggggagggatgggaccccgggggcggggagggggggcaggctgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttccccgcctctcccgccatgcctgctccccgccccagtgttgtgtgaaatcttcggaggaacctgtttacctgttccctccctgcactcctgacccctccccgggttgctgcgaggcggagtcggcccggtccccacatctcgtacttctccctccccgcaggccgctgcgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatcgggggacgcccaagctcagtgcccctccctgagccatgcctcccccaacagctatacgggcagctccccaagttccaggacggagacctcaccctgtaccagtccaataccatcctgcgtcacctgggccgcacccttggtgagtcttgaacctccaagtccagggcaggcatgggcaagcctctgcccccggagcccttttgtttaaatcagctgccccgcagccctctggagtggaggaaactgagacccactgaggttacgtagtttgcccaaggtcaagcctgggtgcctgcaatccttgccctgtgccaggctgcctcccaggtgtcaggtgagctctgagcacctgctgtgtggcagtctctcatccttccacgcacatcctcttcccctcctcccaggctggggctcacagacagccccctggttggcccatccccagtgactgtgtgttgatcaggcgcccagtcacgcggcctgctcccctccacccaaccccagggctctatgggaaggaccagcaggaggcagccctggtggacatggtgaatgacggcgtggaggacctccgctgcaaatacatctccctcatctacaccaactatgtgagcatctgcaccagggttgggcactgggggctgaacaaagaaaggggcttcttgtgccctcaccccccttacccctcaggtggcttgggctgaccccttcttgggtcagggtgcaggggctgggtcagctctgggccaggggcccaggggcctgggacaagacacaacctgcacccttattgcctgggacatcaaccagccaagtaacgggtcatgggggcgagtgcaaggacagagacctccagcaactggtggtttctgatctcctggggtggcgagggcttcctggagtagccagaggtggaggaggatttgtcgccagtttctggatggaggtgctggcacttttagctgaggaaaatatgcagacacagagcacatttggggacctgggaccagttcagcagaggcagcgtgtgtgcgcgtgcgtgtgcgtgtgtgtgcgtgtgtgtgtgtacgcttgcatttgtgtcgggtgggtaaggagatagagatgggcgggcagtaggcccaggtcccgaaggccttgaacccactggtttggagtctcctaagggcaatgggggccattgagaagtctgaacagggctgtgtctgaatgtgaggtctagaaggatcctccagagaagccagctctaaagcttttgcaatcatctggtgagagaacccagcaaggatggacaggcagaatggaatagagatgagttggcagctgaagtggacaggatttggtactagcctggttgtggggagcaagcagaggagaatctgggactctggtgtctggcctggggcagacgggggtgtctcaggggctgggagggatgagagtaggatgatacatggtggtgtctggcaggaggcgggcaaggatgactatgtgaaggcactgcccgggcaactgaagccttttgagaccctgctgtcccagaaccagggaggcaagaccttcattgtgggagaccaggtgagcatctggccccatgctgttccttcctcgccaccctctgcttccagatggacacaggtgtgagccatttgtttagcaaagcagagcagacctaggggatgggcttaggccctctgcccccaattcctccagcctgctcccgctggctgagtccctagcccccctgccctgcagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagctactatgagcactgtgtttcctgggacggggcttaggggttctcagcctcgaggtcggtgggagggcagagcagaggactagaaaacagctcctccagcacagtcagtggcttcctggagccctcagcctggctgtgtttactgaacctcacaaactagaagaggaagaaaaaaaaagagagagagaaacaaagagaaat

SEQ ID NO: 55 - DNA, 2011 bp, Homo sapiens
  misc_feature: B-Actin >gi|28337|emb|Y00474.1|HSACTBPR Human beta-actin gene 5′-flanking region, CpG Island 1656 to 1955
gagctctgtctcttggccagctgaatggaggcccagcggcaacacaggtcctgcctggggatcaggtctgctctgcaccccaccttgctgcctggagccgcccacctgacaacctctcatccctgctctgtagatccggtcccatccccactgcccaccccacccccccagcactccacccagttcaacgttccacgaacccccagaaccagccctcatcaacaggcagcaagaagggccccccgcccatcgccccacaacgccagccgggtgaactgtagcgttggcaggtcctgaggcagctgaaagatacaaggccagggacaggacagtcccatccccaggaggcagggagtatacaggctggggaagtttgcccttgcgtggggtggtgatggaggaggctcagcaagtcttctggactgtgaacctgtgtctgccactgtgtgctgggtggtggtcatctttcccaccaggctgtggcctctgcaaccttcaagggaggagcaggtcccattggctgagcacagccttgtacgtgaactgaacaagcagcctccttcctggccacaggttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccacttccttcccagggctgcggctgggtaggtttgtagccttcatcacgggccacctccagccactggaccgctggcccctgccctgtcctggggagtgtggtcctgcgactctaatggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtcggcgaagccggtgagtgagcggcgcggggccaatcgcgtgcgccgttccgaaagttgccttttatggctcgagcggccgcggcggcgccctataaaacccagcggcgcgacgcgccacc

SEQ ID NO: 56 - DNA, 1792 bp, Homo sapiens
  misc_feature: >gi|5016088|ref|NM_001101.2| Homo sapiens actin, beta (ACTB), mRNA
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctt

SEQ ID NO: 57 - DNA, 2762 bp, Homo sapiens
  misc_feature: RARB2 >gi|14916495|ref|NM_016152.2| Homo sapiens retinoic acid receptor, beta (RARB), transcript variant 2, mRNA
gtgacagaagtagtaggaagtgagctgttcagaggcaggagggtctattctttgccaaaggggggaccagaattccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatgcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaatatgatttacacttgtcaccgagataagaactgtgttattaataaagtcaccaggaatcgatgccaatactgtcgactccagaagtgctttgaagtgggaatgtccaaagaatctgtcaggaatgacaggaacaagaaaaagaaggagacttcgaagcaagaatgcacagagagctatgaaatgacagctgagttggacgatctcacagagaagatccgaaaagctcaccaggaaactttcccttcactctgccagctgggtaaatacaccacgaattccagtgctgaccatcgagtccgactggacctgggcctctgggacaaattcagtgaactggccaccaagtgcattattaagatcgtggagtttgctaaacgtctgcctggtttcactggcttgaccatcgcagaccaaattaccctgctgaaggccgcctgcctggacatcctgattcttagaatttgcaccaggtataccccagaacaagacaccatgactttctcagacggccttaccctaaatcgaactcagatgcacaatgctggatttggtcctctgactgaccttgtgttcacctttgccaaccagctcctgcctttggaaatggatgacacagaaacaggccttctcagtgccatctgcttaatctgtggagaccgccaggaccttgaggaaccgacaaaagtagataagctacaagaaccattgctggaagcactaaaaatttatatcagaaaaagacgacccagcaagcctcacatgtttccaaagatcttaatgaaaatcacagatctccgtagcatcagtgctaaaggtgcagagcgtgtaattaccttgaaaatggaaattcctggatcaatgccacctctcattcaagaaatgctggagaattctgaaggacatgaacccttgaccccaagttcaagtgggaacacagcagagcacagtcctagcatctcacccagctcagtggaaaacagtggggtcagtcagtcaccactcgtgcaataagacattttctagctacttcaaacattccccagtaccttcagttccaggatttaaaatgcaagaaaaaacatttttactgctgcttagtttttggactgaaaagatattaaaactcaagaaggaccaagaagttttcatatgtatcaatatatatactcctcactgtgtaacttacctagaaatacaaacttttccaattttaaaaaatcagccatttcatgcaaccagaaactagttaaaagcttctattttcctctttgaacactcaagatgcatggcaaagacccagtcaaaatgatttacccctggttaagtttctgaagactttgtacatacagaagtatggctctgttctttctatactgtatgtttggtgctttccttttgtcttgcatactcaaaataaccatgacaccaaggttatgaaatagactactgtacacgtctacctaggttcaaaaagataactgtcttgctttcatggaatagtcaagacatcaaggtaaggaaacaggactattgacaggactattgtacagtatgacaagataaggctgaagatattctactttagttagtatggaagcttgtctttgctctttctgatgctctcaaactgcatcttttatttcatgttgcccagtaaaagtatacaaattccctgcactagcagaagagaattctgtatcagtgtaactgccagttcagttaatcaaatgtcatttgttcaattgttaatgtcactttaaattaaaagtggtttattacttgtttaatgacataactacacagttagttaaaaaaaatttttttacagtaatgatagcctccaaggcagaaacacttttcagtgttaagtttttgtttacttgttcacaagccattagggaaatttcatgggataattagcaggctggtctaccactggaccatgtaactctagtgtccttcctgattcatgcctgatattgggatttttttccagcccttcttgatgccaagggctaattatattacatcccaaagaaacaggcatagaatctgcctcctttgaccttgttcaatcactatgaagcagagtgaaagctgtggtagagtggttaacagatacaagtgtcagtttcttagttctcatttaagcactactggaatttttttttttgatatattagcaagtctgtgatgtactttcactggctctgtttgtacattgagattgtttgtttaacaatgctttctatgttcatatactgtttacctttttccatggactctcctggcaaagaataaaatatatttatttt

SEQ ID NO: 58 - DNA, 5487 bp, Homo sapiens
  misc_feature: TIMP3 >gi|21536431|ref|NM_000362.3| Homo sapiens tissue inhibitor of metalloproteinase 3 (Sorsby fundus dystrophy, pseudoinflammatory) (TIMP3), mRNA
tctgtcgacttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctgcaaacagcagatggcttcccatatcccagagagtaagaaccagagagagagagaaagagagagagtttgggtctttctcctctgtgcctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggcgcgccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagcggcgcagagcgggcagcaggcaggcggcgggcgctcagacggcttctcctcctcctcttgctcctccagctcctgctccttcgccgggaggccgcccgccgagtcctgcgccagcgccgaggcagcctcgctgcgccccatcccgtcccgccgggcactcggagggcagcgcgccggaggccaaggttgccccgcacggcccggcgggcgagcgagctcgggctgcagcagccccgccggcggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaacaaaaatgaaaaactactccatttgaggattgtaattcccacccctcttgcttcttccccacctcaccatctcccagaccctcttccctttgcccttctcctccaatacataaaggacacagacaaggaacttgctgaaaggccaaccatttcaggatcagtcaaaggcagcaagcagatagactcaaggtgtgtgaaagatgttatacaccaggagctgccactgcatgtcccaaccagactgtgtctgtctgtgtctgcatgtaagagtgagggagggaaggaaggaactacaagagagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatggggacataagctaatttttttacaggacacagaattctgttcaatgctgttaaatatgccaatagtttaatctcttctattttgttgtcgttgcttgtttgaagaaaatcatgacattccaagttgacatttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttgtagggtttctgttgtgtttttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcagttcattccactttaggaaacagagctgccaattgaaacagaagaagaaaaaaaaaaaaagcagcagacaacacactgtagagtcttgcacacacacaagtgcccaggcaaggtgcttggcagaaccgcagagtgggaagagagtaccggcatcgggtttccttgggatcaatttcattaccgtgtacctttcccattgtggtcatgccatttggcagggggagaatgggaggcttggccttctttgtgaggcagtgtgagcagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcagaaccacatccccatctgttaggccttggtgaggaggccctgggcaaagaagggtctttcgcaaagcgatgtcagagggcggttttgagctttctataagctatagctttgtttatttcacccgttcacttactgtataatttaaaatcatttatgtagctgagacacttctgtatttcaatcatatcatgaacattttattttgctaaatcttgtgtcatgtgtaggctgtaatatgtgtacattgtgtttaagagaaaaatgaaacccacatgccgccattttcctgaatcaaattctgcagtggaatggagaggaaaatacttctaggcaagcagctagactggtgaattgggggaaatagaaggaactagtaactgagactcctccagcctcctccctattggaatcccaatggctcctggagtaggaaaaaagtttaaactacattcatgttcttgttctgtgtcactcggccctgggtagtctaccatttacttcaccccaagtcctgctgcccatccagttgggaagccatgattttcctaagaatccagggccatgggagatacaattccaagttctcgcttcctcctttgggcatctcttctgcctcccaatcaaggaagctccatgctcaggctctcagctctcgggccagtgctctgctctgtccagggtaggtaatactgggagactcctgtcttttaccctcccctcgttccagacctgcctcatggtggcaacatggttcttgaacaattaaagaaacaaatgactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttcactttttatagtgtgtcctttattctaaacagtaaagtggttttatttctatcac

SEQ ID NO: 59 - DNA, 866 bp, Homo sapiens
  misc_feature: APC >gi|551463|gb|U02509.1|HSU02509 Human adenomatous polyposis coli (APC) gene, promoter sequence
  misc_feature (315)..(315): n is a, c, g, or t
acttatatatctgacagttgatttgtcctcacctctaaattggaatttaagcatcacctggttcgatttaatgcaatgtagaatttgcattaaaatactacattaaagcctcagatttgtagtagctaacagcacttctatgtatgtgtcagggactgctctaaatacttcatatatattaactcctctattctgtacttctgttcccgttttatacagcaggaaattgaaacactgagaggttaagtaactaaagttacagagctagagtgacaggagtaaagcttcaactcaggcaacccagacgtccagagntctgatctccactactaagctgctagcatagcttttctggtaactatttttaattcaatataattcgaatgatctatctaacaagtcatcactctgacaactcagtgacttgtaatgtaaaattattcattgtaattcacttaatattattgtttctctgtgctgcaaaaatcatagcaatcgagatgtaatttattactctccctcccacctccggcatcttgtgctaatccttctgccctgcggacctcccccgactctttactatgcgtgtcaactgccatcaacttccttgcttgctggggactggggccgtgagggcatacccccgaggggtacggggctagggctaggcaggctgtgcggttgggcggggccctgtgccccactgcggagtgcgggtcgggaagcggagagagaagcagctgtgtaatccgctggatgcggaccagggcgctccccattcccgtcgggagcccgccgattggctgggtgtgggcgcacgtgaccgacatgtggctgtattggtgcagcccgccagggtgtcactg

SEQ ID NO: 60 - DNA, 10386 bp, Homo sapiens
  misc_feature: >gi|21626462|ref|NM_000038.2| Homo sapiens adenomatosis polyposis coli (APC), mRNA
  misc_feature (9521)..(9521): n is a, c, g, or t
attgaggactcggaaatgaggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagctttacccagcctgtttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaattttttncttcagcttctatgcattaagagtaaaattcctcttactgtaataaaaacaattgaagaagactgttgccacttaaccattccatgcgttggcacttatctattcctgaaattcttttatgtgattagctcatcttgatttttaacatttttccacttaaacttttttttcttactccactggagctcagtaaaagtaaattcatgtaatagcaatgcaagcagcctagcacagactaagcattgagcataataggcccacataatttcctctttcttaatattatagaaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagagaaaacctttttaagcatggtggggcactcagataggagtgaatacacctacctggtggtcat

SEQ ID NO: 61 - DNA, 7273 bp, Homo sapiens
  misc_feature: PTGS2 >gi|3282785|gb|AF044206.1| Homo sapiens cyclooxygenase (COX-2) gene, promoter and exon 1
ggtacccaggctggagtgcactggtgtgatcatagctcactaacctcgaactcctgggcttaggcaatcctcttgccttggcctcccaaagtgccaggattacaggcatgagccaccacagtggagctctcaattctgatactaataatttgtgtcttctctttttttccttagcctgactagagtaattaactttatgtcttttaaaagaaccacctttttggttttacccattttcttttttgattttctgtttttgatttgattgatatctactctaattttttattatttcttttcctctgcttactttgaatttaattacttttcttttttgtagtctcctaaaatagaagcttatattattgattttagatctttcttcttttctattacagcactcaatgctataaatttccctctaagcattgctttcactgcatcctacaatatttcaactctattgttatttagctcaaaagaggttcttaatttctattgggatttctctttgacccatgtgttattcagaagtgttccgtgtgatctccaaatatttgggagtttttcagctatctttctattaatcatttcttgtttaattctattgtggcctgagagcatatattgtatgatttatattcttgtaaatgtgttaaggtgtgtcttatggtgcagaatgcggtttatcttgctatatgttccttagagaataatgtatgttctgctgttattggataaagtagtctatagatgtcagttacatctcgttgattaatggtgctgttgagttcagctatgtcctaaatgattttctgtctgctgtatctgtctatttctgacacaaggctgttgaagtctccaaccataataatgaattaatctatttttctttgcagttttatcaattttgtcttatatatattgatgctccattgtttggcacatacacattaagaattgttatgtcttcttggagaatttacctttccataacatgtaacatttccctttattcctgataatttttcttgctcaaaagtttgccctgttggaaattaccagaactactctggctttatttgattagtgttagcatgctctctctttctctattcttacacttttaatgtatacttgactttgtatttaaagtggggttcttatagaaaacatatacttggtagggtgggaagtaaaataaaaagaaatacttgggtattggtttgatccactctaacaatctctatgttttaattgatgtatttagaccattgatacttatttttttatcctcatccctgtgattacccagagagctgcttaaattgattattgatatagacaaattaataattaatatctaccgtttgttactgttttctatttttcattgcccttactttctgctcctattttttgctcctttttctgttaatttaggttttgagttattttatatcattctattttctctcccttctcagcatatgaattatctttctttttgacttttttagtggctgccctgaaggttgcaatgtacatttacaaccagtcccaatctcctttcaaaaaacacaatactgtttcatggctagtgcaagtacctaataataagaagtcactcctaatttctttctctcattctttgtatctttactgttattcatttcacttgtacataagctgtaatctttcaatacattattgctattattatttcaaaacatgttatctattatatctatttaaaataagaaaaataggccaggtgcagtggcttactcatgtaatcccagcactttgggagaccgatggattgctagagctcaggaattcgagaccagcctgggcaacatagtgaaaccctgtctctactaaaaatacaaaaaaaaaaattgctgggcatggtggcatgggcctgtggtcacagctactcgggaggctgaggtgagaggattgcttgagcctgggaggcagaggttgcagtgaaccaaaatcaagctactgcactccagcctaagtgacagagtgagaccctgtctcaaaaaaaaaatgaaagaattatttttatttatcttcacttatttcttctctaatgctctttgtttctttagtatgtagatccaagtttctaacctgtatcatttttcttatctcaataacttcttttaacatttctcacaaagcagatctactggccacagaatgcctcaattttcatttgtctgagaaaaccttatttctccttcacttttgaaagataattttgtagggtacagaattctaggttgtaggttttttcccctcaaagtgaaatatttcattccactcttttcttctttgtatggtatctgagaagaagtcagatgtaattcttatcattattacttaaaagattgcttctgttcctttctctcttctccttcccttctttccttctctgtatattacaccttttatagttgccccatatttcttagatattatgttttggttttcttctgtgtttttttctttgattctcagttttagaagtctctatttatatatctgcaatcgcagggattctttcctctgccatgtccagtctactaataagcccttacagacattgttgacttctgttccagtgtttttgatctctagcatttctctgattatttcttggaattgccatctgtctacttacattaccaacctattcttgtgtgttgtcttatcatagtaattgcagttgttttaatttcataggtattgtaatttcaacatctctaccatatttgacattgattctgatgcttgctctgtcttatcaagctatgtttttgtcttttagtgtgacttctaattttttgttgaaagccaggcatgatgtactgagtgaaagaaactcaatacattgtaatgtgacgataagagttcaggggaagtgaagcattctatagtcctatagcaggtctcggccttttagtgagcctgtgcctatgaacggtgactttcaacaagtgcttttcattccactcttttcctgtccttaagtgggacaagatcactgggggggggctagaattgggtatttcccttctccaatgtagaagctaaagagagggctggagttgggtatttttcttcccctgtatggaaagctagaggcagttaaatttggatattttccttcttctaattcagttaggctgcgacaaaaatcccgacagtttaggctctaatattataaaataatttctcttgagtataggccttattaagaacactatactctgatggagctgagggggagttttctctgatattcactgcgagaacctcgtagagctccaggaagcaaaactcacaaaagtgtgggagtcttccagaatttttcctttgcagacttatctgcactgaacctccagaaattcatcaattacagttcaggttttcctacccaggtactggttttcatggaggtttctgcctgtgcatttctgctccagtaagttgttcttcttgtatggtctgtctttcaaattttttaagtagggttatgacctgtcgcctcacttctctgacagttctgagagtgttgatttttcagtttgcttagatttttacttgtttttaggatgaagtgacaatttccaagctcctccctgacatgccagatcagaaactgaaagtcctaagcctcatattctgtgcgtgggtatgttcacatcctgcctgctccagtgcccccacctcacactctctttcccttccttgtccccttgtgagatttctaggtccaatacaaagactgtgttcaactcattcaactacttggctcatctgagtattataatgaacaatcacaaaaaaaaatgaagtaaaagaaaaatccatcaaagaattgagatatttgagaaaaagaaaggagatcagtgttttataaaacttagaaatagattttttaagtgtttcttcattgacttatgtgaaaggacttttcttaatttaacaaattatgtgctttcgtttatagcctcaaaacttcttgtgtagctaagaatgggtaaataatcaggctttactaaaggactaacgtaaagatcttctgtaagtaacatttctgctactcaaggaagagataaacttcatggcataaccttgccaaagtatactaagaataaccctgacacaaagctcttttttcagccaacatgccatgaaagaaagaagacaaggggtgatctccactctctaagtgaaccactaaacccaccaaagaagaaacgagggaaatagaaagaggacccttgcctgagataatggatctgtatgtatgagtagtagaaccctgctcaaagtacaaggaagggaaaaaaaagttagtttatttggaattttggacattaagagtctttattgttcattttcttttaactcacatgaatggcttatcacttcaattaataaatatttcatttcttttcaatctatattcatgaaacaaatctgaaatgaacagtgcaacatgtgaatgtttagaacattataaaattaaacacaaaatctgtctggcaatcttcctagcatcttaggaaaaaagttgacaaaatttcaagcagcagaagggggcagtaaaactcaacagaaagctctggaagatttttaagattcttccttattttcttttcatgtagagtatttcccaacaaatttcagacgctaatagaaattttgtacaacagatccatatatttgcctaaaatagacacagaaacattgatatatgcaaacatgagagctataagttttacatgatcaaaaccttttttttatggtacacaatagtcacagtacttttccatataaaacaggtttagtggtcttaatttagtttggcacatttaatacactcccatgaccagcatcccaaatgtacctatccgttttattttattgtctcagaattgtcagttatttaataaattatgtaacttttttccttatgctcagatttgcacttctttctaaaactctgcccatccttaaagtcccagattctccttgaacttttttttttgactttccaagtacatggaactcttcactctatcctgctatataagtgacagaatttccactatgggatagatggagttcaattcctttgagtttaaaataatctaaatataattattccttatgccctgtttttccctcacttttgtatccaaatctcttttcagacaacagaacaattaatgtctgataaggaagacaatgatgatgatcacttcaaaataagcttgaattcaggattgtaatgtaaaattttagtactctctcacagtatggattctaacatggcttctaacccaaactaacattagtagctctaactataaacttcaaatttcagtagatgcaacctactcctttaaaatgaaacagaagattgaaattattaaattatcaaaaagaaaatgatccacgctcttagttgaaatttcatgtaagattccatgcaataaataggagtgccataaatggaatgatgaaatatgactagaggaggagaaaggcttcctagatgagatggaattttagtcatccgtgtctcatgaagaatcagatgtgtacactaagcaaaacagttaaaaaaaaaacctccaagtgagtctcttatttatttttttcttataagacttctacaaattgaggtacctggtgtagttttatttcaggttttatgctgtcattttcctgtaatgctaaggacttaggacataactgaattttctattttccacttcttttctggtgtgtgtgtatatatatatgtatatatacacacacacatatacatatatatatttttagtatctcaccctcacatgctcctccctgagcactacccatgatagatgttaaacaaaagcaaagatgaaattccaactgttaaaatctcccttccatctaattaattcctcatccaactatgttccaaaacgagaatagaaaattagccccaataagcccaggcaactgaaaagtaaatgctatgttgtactttgatccatggtcacaactcataatcttggaaaagtggacagaaaagacaaaagagtgaactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccgcctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtcctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctaccccccgcgccgcgccctgcccgaagctt

SEQ ID NO: 62 - DNA, 4465 bp, Homo sapiens
  misc_feature: >gi|4506264|ref|NM_000963.1| Homo sapiens prostaglandin-endoperoxide synthase 2 (prostaglandin G/H synthase and cyclooxygenase) (PTGS2), mRNA
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 63 - DNA, 1336 bp, Homo sapiens
  misc_feature: 14-3-3-Sigma >gi|45238846|ref|NM_006142.3| Homo sapiens stratifin (SFN), mRNA
gagagacacagagtccggcattggtcccaggcagcagttagcccgccgcccgcctgtgtgtccccagagccatggagagagccagtctgatccagaaggccaagctggcagagcaggccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagaaggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccggggaagaggggggcgaggctccccaggagccccagagctgagtgttgcccgccaccgccccgccctgccccctccagtcccccaccctgccgagaggactagtatggggtgggaggccccacccttctcccctaggcgctgttcttgctccaaagggctccgtggagagggactggcagagctgaggccacctggggctggggatcccactcttcttgcagctgttgagcgcacctaaccactggtcatgcccccacccctgctctccgcacccgcttcctcccgaccccaggaccaggctacttctcccctcctcttgcctccctcctgcccctgctgcctctgatcgtaggaattgaggagtgtcccgccttgtggctgagaactggacagtggcaggggctggagatgggtgtgtgtgtgtgtgtgtgtgtgtgtgtgtgtgtgcgcgcgcgccagtgcaagaccgagattgagggaaagcatgtctgctgggtgtgaccatgtttcctctcaataaagttcccctgtgacactcaaaaaaaaaaaaaaaaaaaaa

SEQ ID NO: 64 - DNA, 1968 bp, Homo sapiens
  misc_feature: RASSF1A >gi|25777678|ref|NM_007182.4| Homo sapiens Ras association (RalGDS/AF-6) domain family 1 (RASSF1), transcript variant A,mRNA
tctcctcagctccttcccgccgcccagtctggatcctgggggaggcgctgaagtcggggcccgccctgtggccccgcccggcccgcgcttgctagcgcccaaagccagcgaagcacgggcccaaccgggccatgtcgggggagcctgagctcattgagctgcgggagctggcacccgctgggcgcgctgggaagggccgcacccggctggagcgtgccaacgcgctgcgcatcgcgcggggcaccgcgtgcaaccccacacggcagctggtccctggccgtggccaccgcttccagcccgcggggcccgccacgcacacgtggtgcgacctctgtggcgacttcatctggggcgtcgtgcgcaaaggcctgcagtgcgcgcattgcaagttcacctgccactaccgctgccgcgcgctcgtctgcctggactgttgcgggccccgggacctgggctgggaacccgcggtggagcgggacacgaacgtggacgagcctgtggagtgggagacacctgacctttctcaagctgagattgagcagaagatcaaggagtacaatgcccagatcaacagcaacctcttcatgagcttgaacaaggacggttcttacacaggcttcatcaaggttcagctgaagctggtgcgccctgtctctgtgccctccagcaagaagccaccctccttgcaggatgcccggcggggcccaggacggggcacaagtgtcaggcgccgcacttccttttacctgcccaaggatgctgtcaagcacctgcatgtgctgtcacgcacaagggcacgtgaagtcattgaggccctgctgcgaaagttcttggtggtggatgacccccgcaagtttgcactctttgagcgcgctgagcgtcacggccaagtgtacttgcggaagctgttggatgatgagcagcccctgcggctgcggctcctggcagggcccagtgacaaggccctgagctttgtcctgaaggaaaatgactctggggaggtgaactgggacgccttcagcatgcctgaactacataacttcctacgtatcctgcagcgggaggaggaggagcacctccgccagatcctgcagaagtactcctattgccgccagaagatccaagaggccctgcacgcctgcccccttgggtgacctcttgtacccccaggtggaaggcagacagcaggcagcgccaagtgcgtgccgtgtgagtgtgacagggccagtggggcctgtggaatgagtgtgcatggaggccctcctgtgctgggggaatgagcccagagaacagcgaagtagcttgctccctgtgtccacctgtgggtgtagccaggtatggctctgcacccctctgccctcattactgggccttagtgggccagggctgccctgagaagctgctccaggcctgcagcaggagtggtgcagacagaagtctcctcaatttttgtctcagaagtgaaaatcttggagaccctgcaaacagaacagggtcatgtttgcaggggtgacggccctcatctatgaggaaaggttttggatcttgaatgtggtctcaggatatccttatcagagctaagggtgggtgctcagaataaggcaggcattgaggaagagtcttggtttctctctacagtgccaactcctcacacaccctgaggtcagggagtgctggctcacagtacagcatgtgccttaatgcttcatatgaggaggatgtccctgggccagggtctgtgtgaatgtgggcactggcccaggttcataccttatttgctaatcaaagccagggtctctccctcaggtgttttttatgaagtgcgtgaatgtatgtaatgtgtggtggcctcagctgaatgcctcctgtggggaaaggggttggggtgacagtcatcatcagggcctggggcctgagagaattggctcaataaagatttcaagatcctcaaaaaaaaaaaaaaaaaaa

SEQ ID NO: 65 - DNA, 17 bp, Artificial
  GSTP1 primer
agttgcgcggcgatttc

SEQ ID NO: 66 - DNA, 23 bp, Artificial
  Probe (5′FAM/3′TAMRA) GSTP1
cggtcgacgttcggggtgtagcg

(19) United States
(12) Patent Application Publication
(10) Pub. No.: US 2007/0059753 A1
(43) Pub. Date: Mar. 15, 2007

(54) Detecting gene methylation

(76) Inventors: Tatiana Vener, Warren, NJ (US); Jyoti Mehrotra, Warren, NJ (US); Shobha Varde, Warren, NJ (US); Abhijit Mazumder, Warren, NJ (US); Jon Baden, Warren, NJ (US); John Backus, Raritan, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/520,233

(22) Filed: Sep. 13, 2006

Related U.S. Application Data
(60) Provisional application No. 60/717,790, filed on Sep. 15, 2005.

(51) Int. Cl. C12Q 1/68

(52) U.S. Cl. 435/6

(57) ABSTRACT

Methods, kits, and compositions for detecting the methylation status of various genes are useful in various diagnostic applications involving suspected proliferative disorders such as prostate cancer.

### CROSS REFERENCE TO RELATED APPLICATION

This application claims the benefit of U.S. Provisional Patent Application Ser. No. 60/717,790 filed Sep. 15, 2005.

### BACKGROUND OF THE INVENTION

This invention relates to the interrogation of methylated genes in applications such as diagnostic and prognostic assays for cellular proliferative disorders such as cancer.

The genes involved include the glutathione-S-transferase (GSTP1) gene or portions of it.

In higher order eukaryotes DNA is methylated only at cytosines located 5′ to guanosine in the CpG dinucleotide. This modification has important regulatory effects on gene expression, especially when it involves CpG rich areas (CpG islands) located in gene promoter regions. Abberant methylation of normally unmethylated CpG islands is a frequent event in immortalized and transformed cells and has been associated with transcriptional inactivation of certain tumor suppressor genes or genes otherwise associated with the amelioration of certain human cancers.

Glutathione S-transferases (GSTs) catalyze intracellular detoxification reactions, including the inactivation of electrophilic carcinogens, by conjugating chemically-reactive electrophiles to glutathione (C. B. Pickett, et al., Annu. Rev. Blocbern., 58:743, 1989; B. Coles, et al., CRC Crit. Rev. Biochem. Mol. Biol., 25:47, 1990; T. H. Rushmore, et al., J. Biol. Chem. 268:11475, 1993). Human GSTs, encoded by several different genes at different loci, have been classified into four families referred to as alpha, mu, pi, and theta (B. Mannervik, et al., Biochem. J., 282:305, 1992). Many human cancers such as prostate and hepatic cancers exhibit decreased GSTP1 expression relative to their tissues of origin.

In the case of prostate cancer, in particular, designing methylation specific PCR (“MSP”) primers and probes that discriminate between benign prostatic hyperplasia and prostate adenocarcinoma has proven to be complex. The best published primer/probe sets typically display up to about 75% sensitivity if one adjusts the assays to be 100% specific and less than 60% specific if one adjusts the assays to be 100% sensitive. A more robust and consistent assay might be obtained with new primer and probe combinations. The present invention fulfills such a need.

### SUMMARY OF THE INVENTION

In one aspect of the invention, a method for detecting a cell proliferative disorder in prostatic tissue or other sample of a subject involves contacting a cellular component with a reagent useful in the amplification and detection of hypermethylated regions of certain genes.

In another aspect of the invention, at least one such hypermethylated gene is GSTP1 which can be assayed in combination with one or more other genes.

In another aspect of the invention, a nucleic acid sample suspected of having methylated target sequences is obtained from a biological sample, treating the sample with a reagent that can prime a portion of the target, amplifying the nucleic acid target, and comparing the degree of methylation of the amplified sample with that of a known normal sample. In yet another aspect of the invention, a sequence that is not likely to be methylated is amplified and detected for comparison with the amplified methylated sequence.

In yet another aspect, the invention provides a method for detecting a cell proliferative disorder. This is done by amplifying a gene whose methylation status is indicative of the cell proliferative disorder. The method of detecting may include contacting a nucleic acid-containing specimen or biological fluid with an agent that modifies unmethylated cytosine, amplifying the nucleic acid in the specimen with oligonucleotide primers or priming sequences that distinguish between modified methylated and nonmethylated nucleic acid, and detecting the methylated nucleic acid based on the presence or absence of amplification products produced during amplification.

The methods of the invention encompass determining a methylation ratio of a sample. The methylation ratio is a ratio between the level of methylation of a Marker (or a region of a Marker) that is hypermethylated in a diseased state relative to the level of methylation of a Marker that is not hypermethylated under the same condition (or relative to a region of the same Marker that is not hypermethylated under the same condition). When the methylation ratio is determined via comparison of methylation of a Marker that is hypermethylated in a diseased state to another Marker that is not, the second Marker can be referred to as a reference Marker.

In another aspect of the invention, methylation ratios are determined via quantitative real time PCR.

In another aspect of the invention, reporter molecules for diagnostic and/or prognostic assays are provided.

In another embodiment, the invention provides a kit useful for the detection of a methylated nucleic acid. The kit includes one or more containers; a first container containing a reagent that modifies unmethylated cytosine and a second container containing a reagent that primes amplification of CpG-containing nucleic acid, wherein the reagent distinguishes between modified methylated and nonmethylated nucleic acid.

In yet another aspect of the invention, the nucleic acid sequence that is detected in the methods of the invention or the kits for practicing them includes a promoter or a portion of a promoter.

In yet another aspect of the invention, methods and kits used to detect methylated portions of certain genes also include steps and/or components for assaying the presence of another nucleic acid. The degree to which the purportedly methylated gene is effected by the treatment (e.g., amplification) is compared to that of the other nucleic acid to determine the extent of methylation of the Marker.

### BRIEF DESCRIPTION OF THE DRAWINGS

FIG. 1 is a graph of the results of MSPCR assays using probes and primers of the prior art.

FIG. 2 is a graph of the results of MSPCR assays using hydrolysis probes and primers according to the invention.

### DETAILED DESCRIPTION OF THE INVENTION

Hypermethylation of certain genes correlates with prostatic carcinogenesis. Nucleic acid sequences comprising such genes or portions of such genes are referred to as Markers in this specification. Markers include nucleic acids comprising the following genes or portions of such genes: GSTP1 (Seq. ID. No. 54), RARB2 (Seq. ID. No. 57), RASSF1A (Seq. ID. No. 64), TIMP3 (Seq. ID. No. 58), APC (Promoter=Seq. ID. No. 59, Gene=Seq. ID. No. 60), beta-Actin (Seq. ID. No.55 and 56), PTGS2 (Promoter=Seq. ID. No.61, Gene=Seq. ID. No. 62) and 14-3-3 sigma (Seq. ID. No.63).

Assays for detecting such hypermethylation include such techniques as MSP and restriction endonuclease analysis. The promoter region is a particularly noteworthy target for detecting such hypermethylation analysis. Sequence analysis of the promoter region of GSTP1 shows that nearly 72% of the nucleotides are CG and about 10% are CpG dinucleotides.

The invention provides a method for determining the methylation status of certain regions of the Markers in a tissue or other biological sample of a subject in which the DNA associated with the proliferative disorder is amplified and detected. Since a decreased level of the protein encoded by the Marker (i.e., less transcription) is often the result of hypermethylation of a particular region such as the promoter, it is desirable to determine whether such regions are hypermethylated. This is seen most demonstrably in the case of the GSTP1 gene. Hypermethylated regions are those that are methylated to a statistically significant greater degree in samples from diseased tissue as compared to normal tissue.

For purposes of the invention, a nucleic acid probe or reporter specific for certain Marker regions is used to detect the presence of methylated regions of the Marker gene in biological fluids or tissues. Oligonucleotide primers based on certain portions of the Marker sequence are particularly useful for amplifying DNA by techniques such as PCR. Any specimen containing a detectable amount of the relevant polynucleotide can be used. A preferred specimen of this invention is tissue of urogenital origin, specifically, prostate tissue. Preferably the sample contains epithelial cells.

Some of the primers/probes or reporter reagents of the invention are used to detect methylation of expression control sequences of the Marker genes. These are nucleic acid sequences that regulate the transcription and, in some cases, translation of the nucleic acid sequence. Thus, expression control sequences can include sequences involved with promoters, enhancers, transcription terminators, start codons (i.e., ATG), splicing signals for introns, maintenance of the correct reading frame of that gene to permit proper translation of the mRNA, and stop codons.

The GSTP1 promoter is an expression control sequence exemplary of a useful Marker. It is a polynucleotide sequence that can direct transcription of the gene to produce a glutathione-s-transferase protein. The promoter region is located upstream, or 5′ to the structural gene. It may include elements which are sufficient to render promoter-dependent gene expression controllable for cell-type specific, tissue-specific, or inducible by external signals or agents; such elements may be located in the 5′ or 3′ regions of the of the polynucleotide sequence.

One method of the invention includes contacting a target cell containing a Marker with a reagent that binds to the nucleic acid. The target cell component is a nucleic acid such as DNA or RNA. The reagents can include probes and primers such as PCR or MSP primers or other molecules configured to amplify and detect the target sequence. For example, the reagents can include priming sequences combined with or bonded to their own reporter segments such as those referred to as Scorpion reagents or Scorpion reporters and described in U.S. Pat. Nos. 6,326,145 and 6,270,967 to Whitcombe et. al. (incorporated herein by reference in their entirety). Though they are not the same, the terms “primers” and “priming sequences” may be used in this specification to refer to molecules or portions of molecules that prime the amplification of nucleic acid sequences.

One sensitive method of detecting methylation patterns involves combining the use of methylation-sensitive enzymes and the polymerase chain reaction (PCR). After digestion of DNA with the enzyme, PCR will amplify from primers flanking the restriction site only if DNA cleavage was prevented by methylation. Exemplary target regions to which PCR primers of the invention are designed include primers which flank the region that lies approximately between −71 and +59 bp according to genomic positioning number of M24485 (Genbank) from the transcription start site of GSTP1.

The method of the invention can also include contacting a nucleic acid-containing specimen with an agent that modifies unmethylated cytosine; amplifying the CpG-containing nucleic acid in the specimen by means of CpG-specific oligonucleotide primers; and detecting the methylated nucleic acid. The preferred modification is the conversion of unmethylated cytosines to another nucleotide that will distinguish the unmethylated from the methylated cytosine. Preferably, the agent modifies unmethylated cytosine to uracil and is sodium bisulfite, however, other agents that modify unmethylated cytosine, but not methylated cytosine can also be used. Sodium bisulfite (NaHSO₃) modification is most preferred and reacts readily with the 5,6-double bond of cytosine, but poorly with methylated cytosine. Cytosine reacts with the bisulfite ion to form a sulfonated cytosine reaction intermediate susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template. Scorpion reporters and reagents and other detection systems similarly distinguish modified from unmodified species treated in this manner.

The primers used in the invention for amplification of a CpG-containing nucleic acid in the specimen, after modification (e.g., with bisulfite), specifically distinguish between untreated DNA, methylated, and non-methylated DNA. In methylation specific PCR (MSPCR), primers or priming sequences for the non-methylated DNA preferably have a T in the 3′ CG pair to distinguish it from the C retained in methylated DNA, and the compliment is designed for the antisense primer. MSP primers or priming sequences for non-methylated DNA usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention are oligonucleotides of sufficient length and appropriate sequence so as to provide specific initiation of polymerization on a significant number of nucleic acids in the polymorphic locus. When exposed to the probes or reporters of the invention, the sequences that are amplified by the primers of the invention reveal methylation status and thus diagnostic information. They are more sensitive and specific than those of the prior art.

The inventive primers are most preferably eight or more deoxyribonucleotides or ribonucleotides capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The priming segment of the primer or priming sequence is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on factors such as temperature, buffer, and nucleotide composition. The oligonucleotide primers most preferably contain about 12-20 nucleotides although they may contain more or fewer nucleotides, preferably according to well known design guidelines or rules.

Primers of the invention are designed to be substantially complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequence(s) to hybridize and permit amplification of the genomic locus.

The primers of the invention are employed in the amplification process. That is, reactions (preferably, an enzymatic chain reaction) that produce greater quantities of target locus relative to the number of reaction steps involved. In a most preferred embodiment, the reaction produces exponentially greater quantities of the target locus. Reactions such as these include the PCR reaction. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized + and − strands containing the target locus sequence. The product of the chain reaction is a discrete nucleic acid duplex with termini corresponding to the ends of the specific primers employed.

The primers of the invention may be prepared using any suitable method, such as conventional phosphotriester and phosphodiester methods including automated methods. In one such automated embodiment, diethylphosphoramidites are used as starting materials and may be synthesized as described by Beaucage, et at. (Tetrahedron Letters, 22:1859-1862, 1981). A method for synthesizing oligonucleotides on a modified solid support is described in U.S. Pat. No. 4,458,066.

Any nucleic acid specimen, in purified or non-purified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA. The DNA or RNA may be single stranded or double stranded. In the event that RNA is to be used as a template, enzymes, and/or conditions optimal for reverse transcribing the template to DNA would be utilized. In addition, a DNA-RNA hybrid containing one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule so that the specific sequence constitutes the entire nucleic acid.

The nucleic acid-containing specimen used for detection of methylated CpG may be from any source such as tissue (particularly, prostate tissue and lymphatic tissue), blood, lymph, urine, and ejaculate and may be extracted by a variety of techniques such as that described by Maniatis, et al. (Molecular Cloning: A Laboratory Manual, Cold Spring Harbor, N.Y., pp 280, 281, 1982).

If the extracted sample is impure, it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical or enzymatic means. One physical method of separating nucleic acid strands involves heating the nucleic acid until it is denatured. Typical heat denaturation may involve temperatures ranging from about 80 to 105° C. for up to 10 minutes. Strand separation may also be induced by an enzyme from the class of enzymes known as helicases or by the enzyme RecA, which has helicase activity, and in the presence of riboATP, is known to denature DNA. Reaction conditions that are suitable for strand separation of nucleic acids using helicases are described by Kuhn Hoffmann-Berling (CSH-Quantitative Biology, 43:63, 1978). Techniques for using RecA are reviewed in C. Radding (Ann. Rev. Genetics, 16:405-437, 1982). Refinements of these techniques are now also well known.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. A molar excess (for genomic nucleic acid, usually about 10⁸:1, primer:template) of the two oligonucleotide primers is preferably added to the buffer containing the separated template strands. The amount of complementary strand may not be known if the process of the invention is used for diagnostic applications, so the amount of primer relative to the amount of complementary strand cannot always be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90-100° C. for up to 10 minutes, preferably from 1 to 4 minutes. After this heating period, the solution is allowed to cool to room temperature, which is preferable for the primer hybridization. To the cooled mixture is added an appropriate agent for effecting the primer extension reaction (the “agent for polymerization”), and the reaction is allowed to occur under conditions known in the art. The agent for polymerization may also be added together with the other reagents if it is heat stable. This synthesis (or amplification) reaction may occur at room temperature up to a temperature at which the agent for polymerization no longer functions.

The agent for polymerization may be any compound or system that will function to accomplish the synthesis of primer extension products, preferably enzymes. Suitable enzymes for this purpose include, for example, E. coli DNA polymerase 1, Klenow fragment of E. coli DNA polymerase I, T4 DNA polymerase, other available DNA polymerases, polymerase muteins, reverse transcriptase, and other enzymes, including heat-stable enzymes (e.g., those enzymes which perform primer extension after being subjected to temperatures sufficiently elevated to cause denaturating). A preferred agent is Taq polymerase. Suitable enzymes will facilitate combination of the nucleotides in the proper manner to form the primer extension products complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Most preferably, the method of amplifying is by PCR. Alternative methods of amplification can also be employed as long as the methylated and non-methylated loci amplified by PCR using the primers of the invention is similarly amplified by the alternative means.

The amplified products are preferably identified as methylated or non-methylated with a probe or reporter specific to the product as described in U.S. Pat. No. 4,683,195 to Mullis et. al., incorporated herein by reference in its entirety. Advances in the field of probes and reporters for detecting polynucleotides are well known to those skilled in the art. Optionally, the methylation pattern of the nucleic acid can be confirmed by other techniques such as restriction enzyme digestion and Southern blot analysis. Examples of methylation sensitive restriction endonucleases which can be used to detect 5′ CpG methylation include SmaI, SacII, EagI, MspI, HpaII, BstUI and BssHII.

The most preferred method of the invention involves establishing a methylation ratio. This can be done by establishing a ratio between the amount of amplified methylated species of Marker attained and the amount of amplified reference Marker or non-methylated Marker region amplified. This is best done using quantitive realtime PCR. Ratios above an established or predetermined cutoff or threshold are considered hypermethylated and indicative of having a proliferative disorder such as cancer (prostate cancer in the case of GSTP1). Cutoffs are established according to known methods in which such methods are used for at least two sets of samples: those with known diseased conditions and those with known normal conditions. The reference Markers of the invention can also be used as internal controls. The reference Marker is preferably a gene that is constitutively expressed in the cells of the samples such as B Actin.

The inventive methods and kits can include steps and reagents for multiplexing. That is, more than one Marker can be assayed at a time.

Since a decreased level of transcription of the gene associated with the Marker is often the result of hypermethylation of the polynucleotide sequence and/or particular elements of the expression control sequences (e.g., the promoter sequence), primers prepared to match those sequences were prepared. Accordingly, the invention provides methods of detecting or diagnosing a cell proliferative disorder by detecting methylation of particular areas within the expression control or promoter region of the Markers. Probes useful for detecting methylation of these areas are useful in such diagnostic or prognostic methods. Preferred molecules for the detection of Markers are shown below. The short name for the Marker gene is shown in parentheses along with the type of detection system employed. Antisense only refers to the orientation of the primer that is so designated in relationship to the priming sequence of the other member of the pair with which it is associated. It is not necessarily antisense with respect to genomic DNA.

SEQ ID NO.1 (GSTP1 SCORPION):
CCCCGAACGTCGACCGCTCGGGG-BHQ-HEG-
CGATTTCGGGGATTTTAGGGCGT
SEQ ID NO.2 (GSTP1 SCORPION Antisense Primer):
AAAATCCCGCGAACTCCCGCC
SEQ ID NO.3 (GSTP1 SCORPION):
CCCGAACGTCGACCGCTTTCGGG-BHQ-HEG-
CGATTTCGGGGATTTTAGGGCGT
SEQ ID NO.4 (GSTP1 SCORPION Antisense Primer):
AAAATCCCGCGAACTCCCGCC
SEQ ID NO.5 (GSTP1 SCORPION):
CGGCGGGAGTTCGCGGGCGCCG-BHQ-HEG-
ACTAAATCACGACGCCGACCGC
SEQ ID NO.6 (GSTP1 SCORPION Antisense Primer):
CGGTTAGTTGCGCGGCGATTTC
SEQ ID NO.7 (GSTP1 SCORPION):
CGGGAGTTCGCGGGTCCCG-BHQ-HEG-ACTAAATCACGACGCCGACCGC
SEQ ID NO.8 (GSTP1 SCORPION Antisense Primer):
CGGTTAGTTGCGCGGCGATTTC
SEQ ID NO.9 (GSTP1 SCORPION):
GTGGTTGATGTTTGGGGTATCAACCAC-BHQ-
HEG_AATCCCACAAACTCCCACCAACC
SEQ ID NO.10 (GSTP1 SCORPION Antisense Primer):
GTGGTGATTTTGGGGATTTTAGGGTGT
SEQ ID NO.11 (GSTP1 SCORPION):
ACCCCAGTGGTTGATGTTTGGGGT-BHQ-HEG-
AATCCCACAAACTCCCACCAACC
SEQ ID NO.12 (GSTP1 SCORPION Antisense Primer):
GTGGTGATTTTGGGGATTTTAGGGTGT
SEQ ID NO.13 (GSTP1 SCORPION):
CCCCACAGGTTGGTGGGAGTTTGTGGGG-BHQ-HEG-
CCCAATACTAAATCACAACACCAACCAC
SEQ ID NO.14 (GSTP1 SCORPION Antisense Primer):
TGGTTAGTTGTGTGGTGATTTTGGGGA
SEQ ID NO.15 (GSTP1 SCORPION):
CCCCGAACGTCGACCGCTCGGGG-BHQ-HEG-
CGATTTCGGGGATTTTAGGGCGT
SEQ ID NO.16 (GSTP1 SCORPION Antisense Primer):
AAAATCCCGCGAACTCCCGCC
SEQ ID NO.17 (GSTP1 SCORPION):
CGCACGCCGAACGTCGACCGCAAACGTGCG-BHQ-HEG-
CGATTTCGGGGATTTTAGGGCGT
SEQ ID NO.18 (GSTP1 SCORPION Antisense Primer):
AAAATCCCGCGAACTCCCGCC
SEQ ID NO.19 (GSTP1 SCORPION):
CGCACGGCGAACTCCCGCCGACGTGCG BHQ-HEG-
TGTAGCGGTCGTCGGGGTTG
SEQ ID NO.20 (GSTP1 SCORPION Antisense Primer):
GCCCCAATACTAAATCACGACG
SEQ ID NO.21 (GSTP1 SCORPION):
CCGACGCACAAAAAAACACCCTAAAATCCGTCGG-BHQ-HEG-
GGTTAGTTGTGTGGTGATTTT
SEQ ID NO.22 (GSTP1 SCORPION Antisense Primer):
CACAACACCAACCACTCTTC
SEQ ID NO.23 (GSTP1 TAQMAN PRIMER):
CGTGATTTAGTATTGGGGCGGAGCGGGGC
SEQ ID NO.24 (GSTP1 TAQMAN PRIMER):
ATCCCCGAAAAACGAACCGCGCGTA
SEQ ID NO.25 (GSTP1 TAQMAN PROBE):
TCGGAGGTCGCGAGGTTTTCGTTGGA
SEQ ID NO.26 (RARB2 SCORPION):
GCCGCCGTCGAGAACGCGAGCGGGCGGC-BHQ-HEG-
TACCCCGACGATACCCAAAC
SEQ ID NO.27 (RARB2 SCORPION Antisense Primer):
GGGATTAGAATTTTTTATGCGAGTTGT
SEQ ID NO.28 (RARB2 SCORPION):
CGGCAGGATTGGGATGTCGAGCTGCCG-BHQ-HEG-
TACCCCGACGATACCCAAAC
SEQ ID NO.29 (RARB2 SCORPION Antisense Primer):
GGGATTAGAATTTTTTATGCGAGTTGT
SEQ ID NO.30 (RASSF1A SCORPION):
GCCGCGGTTTCGTTCGGTTCGCGGC-BHQ-HEG-
CCCGTACTTCGCTAACTTTAAACG
SEQ ID NO.31 (RASSF1A SCORPION Antisense Primer):
GCGTTGAAGTCGGGGTTC
SEQ ID NO.32 (TIMP3 SCORPION):
GCGGCGAGTTCGGGTTGTAGCGCCGC-BHQ-HEG-
CGCCTCTCCAAAATTACCGTAC
SEQ ID NO.33 (TIMP3 SCORPION Antisense Primer):
GCGTCGGAGGTTAAGGTTGTT
SEQ ID NO.34 (APC SCORPION):
GCCGGCGGGTTTTCGACGGGCCGGC-BHQ-HEG-
CGAACCAAAACGCTCCCCA
SEQ ID NO.35 (APC SCORPION Antisense Primer):
GTCGGTTACGTGCGTTTATATTTAG
SEQ ID NO.36 (ACTIN SCORPION):
GCGCCCAACCGCACAAGGGCGC-BHQ-HEG-
GGGTATATTTTCGAGGGGTACG
SEQ ID NO.37 (ACTIN SCORPION Antisense Primer):
CGACCCGCACTCCGCAAT
SEQ ID NO.38 (ACTIN SCORPION):
CCGCGCATCACCACCCCACACGCGCGG-BHQ-HEG-
GGAGTATATAGGTTGGGGAAGTTTG
SEQ ID NO.39 (ACTIN SCORPION Antisense Primer):
AACACACAATAACAAACACAAATTCAC
SEQ ID NO.40 (ACTIN SCORPION):
CCCGGCTAAACCCACCATCCAGCCGGG-BHQ-HEG-
GGGAGGGTAGTTTAGTTGTGGTT
SEQ ID NO.41 (ACTIN SCORPION Antisense Primer):
CAAAACAAAAAAACTAAATCTACACAACC
SEQ ID NO.42 (ACTIN SCORPION):
CCGCGGAACATTCAACTCAACCGCGG-BHQ-HEG-
GGAGGAGGAAGGTAGGTTTTT
SEQ ID NO.43 (ACTIN SCORPION Antisense Primer):
ACATACAACAATCAATAACATAAAACCAC
SEQ ID NO.44 (PTGS2/COX2 SCORPION):
CACGCCGCCGTATCTAGGCGTG-BHQ-HEG-
GTTTGTTTCGACGTGATTTTTTCGA
SEQ ID NO.45 (PTGS2/COX2 Antisense Primer):
GCAAAAAATCCCCTCTCCCGC
SEQ ID NO.46 (PTGS2/COX2 SCORPION):
GCCGCGCACAAATTTCCGCGGC-BHQ-HEG-
GAATTGGTTTTCGGAAGCGTTCG
SEQ ID NO.47 (PTGS2/COX2 Antisense Primer):
CCCGAATTCCACCGCC
SEQ ID NO.48 (PTGS2/COX2 SCORPION):
GGCGGAACGCACAAATTTCCGCC-BHQ-HEG-
GAATTGGTTTTCGGAAGCGTTCG
SEQ ID NO.49 (PTGS2/COX2 Antisense Primer):
CCCGAATTCCACCGCC
SEQ ID NO.50 (PTGS2/COX2 SCORPION):
TGCCGCCGCCGTATCTAATGGCGGCA-BHQ-HEG-
GTTTGTTTCGACGTGATTTTTTCGA
SEQ ID NO.51 (PTGS2/COX2 Antisense Primer):
GCAAAAAATCCCCTCTCCCGC
SEQ ID NO.52 (14-3-3 SCORPION):
CGGCCTTCGCTCTTCGCAAAAGGCCG-BHQ-HEG-
GGTAGTTTTTATGAAAGGCGTCGTG
SEQ ID NO.53 (14-3-3 Antisense Primer):
CTAACCGCCCACCACGTT

BHQ = Black Hole Quencher (BioSearch Technologies, San Fransisco, CA)
HEG = Hexaethylene glycol

The kits of the invention can be configured with a variety of components provided that they all contain at least one primer or probe of the invention or a detection molecule (e.g., Scorpion reporter). In one embodiment, the kit includes reagents for amplifying and detecting hypermethylated Marker segments. Optionally, the kit includes sample preparation reagents and/or articles (e.g., tubes) to extract nucleic acids from samples.

In a preferred kit, reagents necessary for one-tube MSP are included such as, a corresponding PCR primer set, a thermostable DNA polymerase, such as Taq polymerase, and a suitable detection reagent(s) such as hydrolysis probe or molecular beacon. In optionally preferred kits, detection reagents are Scorpion reporters or reagents. A single dye primer or a fluorescent dye specific to double-stranded DNA such as ethidium bromide can also be used. The primers are preferably in quantities that yield high concentrations. Additional materials in the kit may include: suitable reaction tubes or vials, a barrier composition, typically a wax bead, optionally including magnesium; necessary buffers and reagents such as dNTPs; control nucleic acid(s) and/or any additional buffers, compounds, co-factors, ionic constituents, proteins and enzymes, polymers, and the like that may be used in MSP reactions. Optionally, the kits include nucleic acid extraction reagents and materials.

### EXAMPLES

### Example 1

### Comparative

A hydrolysis probe assay was designed based upon the primers and probes in the literature and directed to the quantitative assay for detecting prostate cancer in patients with clinically localized disease. This assay includes a core CpG promoter region used to discriminate between neoplastic and non-neoplastic prostate tissue.

The primer and dual-labeled hydrolysis probe sequences tested for this design were as follows:

Forward primer

(Seq. ID. No.65)

| | |
|---|---|
| GSTP1;(−192)MU17 | AGTTGCGCGGCGATTTC |
| Reverse primer | |

(Seq. ID. No.20)

| | |
|---|---|
| GSTP1;(−74)ML22 | GCCCCAATACTAAATCACGACG |
| Probe (5′FAM/3′TAMRA) | |

(Seq. ID. No.66)

| | |
|---|---|
| GSTP 1;(−152)MP23 | CGGTCGACGTTCGGGGTGTAGCG |

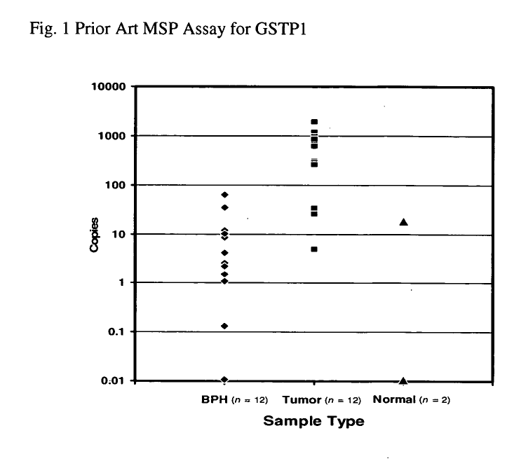

GSTP1 methylation levels were analyzed in 12 samples from benign prostatic hyperplasia (BPH), 12 samples from clinically localized prostate adenocarcinoma (Tumor), and 2 normal samples. Data is presented in total copies of GSTP1 detected and is shown in FIG. 1. The assay was found to display 75% sensitivity for detecting prostate adenocarcinoma when a cutoff was set to the highest copy number exhibited by a benign sample. The moderate distribution of GSTP1 methylation levels in prostate tissues displaying benign prostatic hyperplasia and clinically localized prostate adenocarcinoma suggests an assay that would not be clinically relevant without being complemented by another marker.

### Example 2 (Hydrolysis Probe Assay According to the Invention)

MSP primers and probe were designed to further improve sensitivity and specificity for the GSTP1 assay and were tested on the same sample set as in Example 1. This design was located further downstream of the CpG island and encompassed part of the core promoter and exon 1. The primer and dual-labeled hydrolysis probe sequences tested for this example are as follows:

Forward primer:

(Seq. ID No.23)

| | |
|---|---|
| (−71)MU29 | CGTGATTTAGTATTGGGGCGGAGCGGGGC |
| Reverse primer: | |

(Seq. ID No.24)

| | |
|---|---|
| (+59)ML25 | ATCCCCGAAAAACGAACCGCGCGTA |
| Probe (5′FAM/3′TAMRA) | |

(Seq. ID No.25)

| | |
|---|---|
| GSTP1;(-23)MP26 | TCGGAGGTCGCGAGGTTTTCGTTGGA |

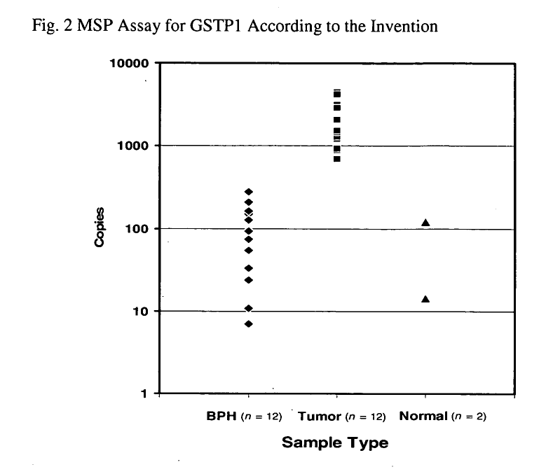

Data is presented in total copies of GSTP1 detected and is shown in FIG. 2. The assay was found to display 100% sensitivity and 100% specificity for detecting prostate adenocarcinoma when a cutoff is set to the highest copy number exhibited by a benign sample.

The 1.5 kb CpG island of GSTP1 spans through the promoter into exon 3 and has been shown to be extensively methylated throughout. Two transcription binding sites AP1 and Sp1 are located just upstream of exon 1 and were the focus of the design of the probes and primers used in this example due to the high content of CpG's within SP1. This proved to be a good region for assay design due to competition between transcription factors and methyl-CpG binding proteins binding to the AP1 and Sp1 sites in order to induce chromatin condensation and gene silencing.

### Example 3

### Scorpion MSP

Scorpion reagents were designed for the following Markers and reference Markers:
Seq. ID No. 19, 20 (GSTP1)
Seq. ID No. 38, 39 (Actin)
Seq. ID No. 28, 29 (RARB2)
Seq. ID No. 30, 31 (RASSF1A)
Seq. ID No. 32, 33 (TIMP3)
Seq. ID No. 34, 35 (APC)
Seq. ID No. 50, 51 (PTGS2)
Seq. ID No. 52, 53 (14-3-3 sigma)

The designs were tested for secondary structures and inappropriate dimerizations using Oligo6 primer design software (Molecular Biology Insights, Cascade, Colo., USA) and Visual OMP (DNA Software, Inc., 120½ West Washington Street Ann Arbor, Mich., USA). Scorpion folding and amplicon probing was modeled using the DNA mfold program (http://bioinfo.math.rpi.edu/˜mfold/dna/form1.cgi) and Visual OMP (DNA Software, Inc., 120½ West Washington Street Ann Arbor, Mich., USA). Scorpions and primers were synthesized in an automated DNA synthesizer using standard phosphoramidite chemistry (Scandinavian Gene Synthesis AB, Koping, Sweden) and Biosearch Technologies (Novato, Calif., USA).

### Example 3

### Scorpion Reagent Testing

The reagents described in Example 2 were tested on various human DNA samples. These samples included commercially available methylated (all cytosines preceding guanine are methylated at position 5) and unmethylated human genomic DNA (Chemicon, Temecula, Calif., USA) as positive and negative template respectively. Genomic DNA from human breast cancer cell line, MCF-7 and colorectal cancer cell line, HCT116 as positive and negative controls for GSTP1 methylation were also used (American Type Culture Collection, Manassas, Va., USA).

Genomic DNA was modified using a commercially available sodium bisulfite conversion reagent kit (Zymo Research, Orange, Calif., USA). This treatment converted all Cytosines in unmethylated DNA into Uracil, whereas in methylated DNA only cytosines not preceding guanine were converted into Uracil. All cytosines preceeding guanine (in a CpG dinucletide) remained as cytosine.

Sodium bisulfite modified genomic DNA (100-150 ng) was amplified in a 25 μl reaction containing the following components: 67 mM Tris pH 8.8, 16.6 mM (NH₄)₂SO₄, 6.7 mM MgCl₂, 10 mM beta mercaptoethanol; 1.25 mM each dATP, dCTP, dGTP, dTTP, 1 U Hot start Taq DNA Ploymerase, 250 nM Scorpion probe, 250 nM reverse or forward primer (depending on scorpion design), 625 nM of passive reference dye, ROX (for ABI7900 runs only).

The samples were then tested in a quantitative real-time PCR assay on an ABI 7900HT sequence detection system and/or Cepheid SmartCycler® PCR instrument.

The PCR conditions used were:

For ABI 7900 runs:
95° C. for 5 mins.; then 40 cycles of 95° C. for 30 sec, 59° C. for 30 sec and 72° C. for 30 sec; and a final extension at 72° C. for 5 mins. Optical data was collected at 59° C. for every cycle.

For Cepheid smart cycler runs:
95° C. for 60 sec; then 40 cycles of 95° C. for 30 sec, 59° C. for 30 sec, and a final extension at 72° C. for 5 min. Optical data was collected at 59° C. for every cycle.

The Scorpion reagents showed a PCR efficiency of 91.2% in the case of GSTP1 reagents and 95.2% in the case of Actin reagents. The average Ct values were:

| | |
|---|---|
| GSTP1 | 28 |
| B Actin | 30 |
| APC | 29 |
| TIMP3 | 30 |
| RASSF1A | 29 |
| RARB2 | 29 |
| PTGS2 | 33 |
| 14-3-3 S | 32 |

### Example 4

### Comparison with Molecular Beacons

Molecular beacons were obtained for detecting GSTP1 hypermethylation along the portion of the gene described in Example 1. The beacons were applied to PCR reactions involving samples as described in Example 2. In each case, B Actin was co-amplified so that methylation ratios could be obtained. Both GSTP1 and B-actin Scorpion probe-primer sets displayed better performance in detection of methylated GSTP1 DNA and unmethylated B Actin DNA. The Ct threshold values for the designed Scorpion sets were more than 1.5 Ct better than for the molecular beacon probes (average Ct of 28.82 for beacons v/s 27.30 for Scorpion reporters). Results are as follows:

| | Universal Methylated DNA, ng | | | | | |
|---|---|---|---|---|---|---|
| | 500 | 250 | 100 | 50 | 10 | 5 |
| Scorpion (GSTP1/Bactin | 272 | 310 | 285 | 296 | 339 | 288 |
| copy# ratio) | | | | | | |
| Mol Beacon (GSTP1/Bactin | 759 | 735 | 758 | 767 | 797 | 814 |
| copy# ratio) | | | | | | |

### Example 5

### Methylation Ratio Cutoffs

Prostate tissue samples from patients with known clinical conditions were then subjected to PCR as described in Example 2. Methylation ratios were calculated and are shown below. Data was analyzed by using the following procedure. Prior to employing an algorithm, the following pre-requisites were followed.
For Actin: CT values<40 for both replicates (as defined by real-time PCR equipment)
For “methylated” GSTP1 result: CT values<40 for both replicates (as defined by real-time PCR equipment)
The slope of both standard curves above −4 (PCR efficiency>80%)
R2: 0.99, 4 relevant data points on the standard curve
Positive assay control has to generate a positive result
Routinely included no-template controls (standard curves) have to be negative

If the pre-requisites were met, copy numbers were calculated for each of the genes (GSTP1 and Actin) based on the standard curves and a ratio was calculated for each sample (including the positive control) by using the following formula: (GSTP1 Copy Number/β-Actin Copy Number)*1000. A cutoff was then established by using a ratio which generated high specificity and high sensitivity. The cutoff was 10 for GSTP1 to provide a sensitivity of 87% and a specificity of 100% for 142 formalin fixed paraffin embedded prostate tissues. Ratios generated were used to determine methylation status of a given sample by employing the following criteria.

| | copy# | copy# | |
|---|---|---|---|
| Results | (mGSTP1) | (B-Actin) | Ratio × 1000 |
| Methylated GSTP1 | >2 | >2 | >10 |
| UnMethylated GSTP1 | any | >200 | <10 |
| Undetermined | <2 | <200 | |

One skilled in the art will recognize that the cutoff can be adjusted, preferably to set the sensitivity and specificity values to those suitable for the purpose for which they will be used (e.g., screening, therapy monitoring, reflex testing of ambiguous diagnoses).

### Example 6

### Combination Markers (Prophetic)

Scorpion reagents for the following combinations are prepared as described in Example 1: GSTP1 Marker and RARβ2, GSTP1 and APC, and GSTP1 and PTGS2. The Markers are run in combination as multiplexed PCR according to Example 2 using repetitive runs of 50 prostate tissue samples with known conditions (i.e., normal, benign hyperplasia, prostate cancer). Expected results are shown below:

| | Seq. ID | | |
|---|---|---|---|
| Marker/s | No. | Sensitivity | Specificity |
| GSTP1 + | | 90% | 100% |
| RARβ2 | | | |
| GSTP1 + | | 96.2% | 92.9 |
| APC | | | |
| GSTP1 + | | 96.2 | 100 |
| PTGS2 | | | |

We claim:

1. A method of diagnosing the presence or predicting the course of a proliferative disorder comprising determining the methylation status of a Marker in a biological sample with the use of a reagent selected from the group consisting of: Seq. ID NO. 1-35 and 44-53.

2. The method according to claim 1 further comprising measuring the presence of a reference Marker.

3. The method according to claim 2 wherein the reference Marker is selected from the group consisting of B Actin.

4. The method of claim wherein a combination of Markers are used.

5. The method of claim 4 wherein the combination of Markers includes a Marker for GSTP1 and a Marker for APC, TIMP, RASSF1A, RARB2, PTGS2, or 14-3-3.

6. The method according to claim 4 wherein reagents for the Markers include a member of the group consisting of Seq. ID. No. 1-35 and 44-53.

7. The method of claim 1 wherein the sample is prostate tissue.

8. The method of claim 1 wherein the sample is urine, serum, plasma, or circulating cells.

9. A composition comprising a member of the group consisting of Seq. ID No. 1-43.

10. A composition comprising a mixture of more than one composition of claim 10.

11. A kit for conducting an assay according to claim 1, comprising: nucleic acid amplification and detection reagents.

12. The kit of claim 11 wherein the amplification and detection reagents are selected from the group consisting of Seq. ID No. 1-43.

13. The kit of claim 12 wherein the amplification and detection reagents are selected from Seq. ID No. 1-25 together with reagents selected from the group consisting of Seq. ID No. 36-43.

14. The kit of claim 11 further comprising instructions.

15. The kit of claim 11 wherein the reagents detect the hypermethylation of a gene selected from the group consisting of GSTP1 and APC, TIMP, RASSF1a, RARB2, PTGS2, or 14-3-3.

16. The kit of claim 11 further comprising reagents for amplifying and detecting the presence of a constitutively expressed gene.

17. The kit of claim 11 further comprising reagents for amplifying and detecting the presence of constitutively expressed genes.

18. The method of claim 1 further comprising establishing a methylation ratio and determining whether the methylation ratio exceeds a cutoff value.

19. The method of claim 1 for use in therapy monitoring.

20. The method of claim 1 for use in screening.

21. The method of claim 1 for use in resolving ambiguous or indeterminate results of cancer assays obtained using methods different from claim 1.
